(19) United States
(12) Patent Application Publication
(10) Pub. No.: US 2019/0177681 A1
(43) Pub. Date: Jun. 13, 2019

(54) AEROBIC MICROORGANISM LARGE-SIZE FERMENTATION PILE TREATMENT SYSTEM CONSIDERING BOTH VENTILATION AND HEAT PRESERVATION AND FERMENTATION FURNACE CAPABLE OF CONTINUOUSLY OPERATING

(71) Applicant: BEIJING CLEAN WATER CULTURE DEVELOPMENT CO., LTD., BEIJING (CN)

(72) Inventor: Hongbin ZHANG, BEIJING (CN)

(73) Assignee: BEIJING CLEAN WATER CULTURE DEVELOPMENT CO., LTD.

(21) Appl. No.: 16/278,733

(22) Filed: Feb. 19, 2019

Related U.S. Application Data
(63) Continuation of application No. PCT/CN2017/099859, filed on Aug. 31, 2017.

(30) Foreign Application Priority Data
Aug. 31, 2016 (CN) 201610796125.9
Mar. 10, 2017 (CN) 201720230137.5
Aug. 29, 2017 (CN) 201721089885.2

(51) Int. Cl. C12M 1/00; C12M 1/34

(52) U.S. Cl. CPC C12M 29/20; C12M 23/38; C12M 41/12

(57) ABSTRACT

The disclosure relates to the technical field of solid aerobic microorganism fermentation, and particularly discloses a microorganism fermentation method and fermentation device. The method comprises the following steps: providing a suspended, space under a fermentation pile; stacking the fermentation materials on the ventilating passage to build the fermentation pile; forming airflow through holes in the material pile and communicating the airflow through holes with the ventilating passage; fermenting the materials to produce heat so as to automatically drive external air to supply the fermentation pile with oxygen. The fermentation method and the fermentation device of the disclosure achieve control on the whole fermentation progress through the drilling sequence of the airflow through holes. Through the microorganism fermentation method of the disclosure, the fermentation pile can gain sufficient oxygen supplementation without an external force, thereby quickly reaching high-temperature fermentation and accelerating a material fermentation speed.

### CROSS-REFERENCE TO RELATED APPLICATIONS

This application is a continuation of International Patent Application No. PCT/CN2017/099859 with a filing date of Aug. 31, 2017, designating the United States, now pending, and further claims priority to Chinese Patent Application No. 201610796125.9 with a filing date of Aug. 31, 2016, Chinese Patent Application No. 201720230137.5 with a filing date of Mar. 10, 2017, and Chinese Patent Application No. 201721089885.2 with a filing date of Aug. 29, 2017. The content of the aforementioned applications, including any intervening amendments thereto, are incorporated herein by reference.

### TECHNICAL FIELD

The present application belongs to the technical field of solid-state aerobic microorganism fermentation, particularly relates to a microorganism fermentation method and fermentation device considering both ventilation and heat preservation, and more particularly relates to an aerobic microorganism fermentation furnace capable of continuously operating and a treatment system of a microorganism fermentation pile for large-scale organism solid wastes.

### BACKGROUND OF THE PRESENT INVENTION

Besides material components and strains of microorganisms, factors restricting a fermentation speed of aerobic microorganisms are temperature, oxygen and water. In a general solid-state fermentation technology heat preservation and, ventilation and oxygenating are a pair of conflicts, a material on the surface can obtain sufficient oxygen, but cannot achieve high temperature fermentation due to rapid, heat dissipation. The material in the core is good in heat preservation performance, but oxygen is insufficient. If a heat preservation layer is added to a fermentation pile at the periphery, it means that oxygen supply is hindered, and the interior is more lack of oxygen. Some methods are that transverse and vertical solid pipes are embedded in the fermentation pile, or an external force is increased for forced ventilation, the solid pipes bring inconvenience for operation of the fermentation pile, ventilation via the external force difficultly reaches optimum air volume neither, and the solution effect is not significant and uneconomic. In general, the solid-state fermentation method requires no more than 65% of the water content of the material, while some flow-state sludge or feces itself contains quite high water content and cannot be formed by stacking and cannot start fermentation neither. Water is regulated through addition of other materials, the quantity of the required materials is, huge, even an improper carbon/nitrogen ratio is caused, and thus difficulty is extremely large.

The common aerobic fermentation method is that the fermentation pile is stacked on the ground, or fermentation is performed in a container. However, in order to supplement oxygen and homogenize fermentation, it is necessary to cooperate with pile turning or stirring, and one fermentation pile is fermented once, and new fermentation materials cannot be added at any time. Thus, this method has no continuous operating capability.

In addition, the size of the fermentation pile is restricted by ventilation conditions and cannot be too large, which is a great restriction factor for the existing huge large-scale solid wastes (such as a rubbish hill) treatment. Furthermore, for organic solid wastes containing viscous hard blocks, it is needed to cut them as pretreatment before fermentation, so as to improve the efficiency and effect of the fermentation. However, after being cut in a common manner, these hard blocks are often easily adhered together and then cannot be sufficiently mixed with auxiliary fermentation materials. After fermentation of other materials is completed, these hard blocks are still remaining, thereby bring inconvenience of operation of subsequent procedures.

### SUMMARY OF PRESENT INVENTION

According to one aspect of the present application, in order to solve the conflict of microorganism fermentation in ventilation and heat preservation, provided is an aerobic microorganism fermentation method considering both ventilation and heat preservation, comprising the following steps:

providing a suspended space under a fermentation pile in advance so that fermentation materials are lifted above the ground, wherein, the suspended space, as a ventilating passage, is communicated with external air;

stacking the fermentation materials on the ventilating passage to build a fermentation pile; making a plurality of up and down penetrated vertical airflow through holes on the material pile, wherein, the upper parts of the vertical airflow through holes are communicated with the outside, and the lower parts thereof are communicated with the ventilating passage; the vertical airflow through holes are formed by downwardly drilling up and down penetrated holes from the top of the fermentation pile by virtue of a tool, or a pipe with grids or ventilating gaps on a pipe wall is pre-embedded, such as a floral pipe for digging a well; and

fermenting the materials to produce heat so as to automatically drive external air to supply the fermentation pile with oxygen, wherein, the required air volume is, automatically regulated without an external energy source so that the fermentation pile is stable in a high-temperature fermentation state.

Preferably, the aerobic microorganism fermentation method considering both ventilation and heat preservation further comprises: achieving control on the whole fermentation progress through the drilling sequence of the vertical airflow ventilating holes, including timing, a pore diameter, quantity and location.

Preferably, the aerobic microorganism fermentation method considering both ventilation and heat preservation further comprises: providing, a heat preservation layer outside the fermentation pile, so as to facilitate preservation of heat to achieve high temperature treatment without affecting supply of oxygen.

Preferably, the fermentation pile is also internally provided with transverse airflow through holes which perpendicularly penetrate through the vertical airflow through holes, so as to enhance the diffusion effect of the entered air; the transverse airflow through holes are achieved by horizontally laying plant ventilating materials at different layer heights of the fermentation pile, or by drilling holes crossed and communicated with or close to the vertical airflow through holes with a tool in a horizontal direction.

Preferably, the aerobic microorganism fermentation method considering both ventilation and heat preservation further comprises a fermentation and treatment method of a diluted material which is difficult to shape due to too much water, the fermentation and treatment method particularly comprising: on the basis of the above ventilating passage structure, providing a wall at the periphery of the fermentation pile to fix the diluted material so that excess water is rapidly permeated to the ventilating passage below to achieve a proper water content, thereby rapidly starting the fermentation of the material. The water permeated to the ventilating passage is collected through arrangement of a coffer and a colleting pool on the ground to supplement water to the fermentation pile or soak the auxiliary fermentation material for absorbing water in the later period of fermentation to participate in subsequent batch fermentation.

In the disclosure, the activity of microorganisms warms up the material, air in the vertical hole is heated and rises to drive external air to be supplemented from the bottom of the fermentation pile to enter the vertical hole, so that oxygen is continuously and evenly supplied to various parts of the fermentation pile. Air supplementation speed is in an automatic regulation relationship with the fermentation temperature. When fermentation starts, the temperature is low and air supplementation speed is slow, facilitating the heat preservation of the fermentation pile; the temperature rises as soon as possibly. With the increase of fermentation temperature, air supplementation is accelerated enough to meet a need of oxygen required by the activity of fermenting a large amount of microorganisms at high temperature. When the temperature is over high to inhibit the activity of the microorganisms, generation of heat by fermentation is inhibited as well. The fermentation temperature is reduced, air supplementation becomes slow, the organisms are active again, and air supplementation is accelerated again. In summary, supply of oxygen is always automatically adaptive to the activity intensity of microorganisms to be regulated. Drilling follows a certain sequence, and the interior of the newly built fermentation pile contains oxygen, drilling is not performed at first, and the fermentation pile preferably accumulates heat; when the temperature starts rising, one part of holes are drilled, the temperature is raised again, and then the pore diameter or quantity of the airflow through hole is enlarged. At the stage of temperature rising, the degree of fermentation at the low-temperature position is poor, and increase of the airflow through holes in this area can accelerate fermentation in this area. The whole control of fermentation is achieved through the drilling sequence. The disclosure solves a conflict between oxygen supply and heat preservation of the fermentation pile using a completely nature method so that the fermentation pile achieves an optimal system balance and can rapidly reach high temperature fermentation, thereby accelerating the treatment speed, reducing the treatment time, at the same time, rapidly the fermentation of the extremely diluted material, and reducing the difficulty of regulating and controlling the water content in the general solid fermentation method.

According, to another aspect of the present application, provided is an aerobic microorganism fermentation furnace capable of continuously operating, comprising a top cover, a fermentation room, a grate and a discharging room, wherein, the vertical surfaces of the fermentation room are sealed, the upper part of the fermentation room is opened, the fermentation materials are put from the upper part and form the fermentation pile therein; the fermentation pile is internally provided with several airflow through holes communicated with the discharging room below, and the grate is arranged between the fermentation room and the discharging room, is of a grid, structure and is used for blocking the materials in the fermentation room above not to drop; this fermentation furnace further comprises a furnace hook for discharging; the loose fermented materials are scraped with the furnace hook along the grid seams of the grate when it is needed for discharging so that the materials on the bottom layer drop into the discharging room, hereby, the aerobic microorganism fermentation furnace capable of continuously operating, in which materials are fed from the upper part and discharged from, is formed.

As an improvement of the above technical solution, one side of the discharging room is provided with a discharging hole; the discharging hole upwardly extends from the bottom of the, discharging room over the grate to a space above the bottom of the fermentation room; the discharging hole is equipped with a flapper; after the flapper is opened, a fermentation situation at the bottom of the fermentation room is checked above the grate and a material which is large in caking ability and difficult to drop is subjected to assistant treatment; discharging is performed under the grate, and after discharging, the flapper is properly sealed, so as to achieve a function of regulating the input of air from the lower part; the bottom surface of the discharging room is inclined at a certain angle from the inner to the outer, and a water colleting tank is arranged outside the discharging room and used for collecting water permeated from the fermentation pile; the discharging room is also internally provided with a temporary heater for heating air entering the fermentation pile in cold winter until the indoor temperature is reached, and the rapid starting and rapid fermentation of the fermentation pile and rapid fermentation are promoted.

More further, the temporary heater can adopt water heating, electric heating and the like and can be placed on a slide trolley so as to facilitate movement and disassembly. A shelter is placed on the temporary heater to avoid the fermentation material to drop into the device; a part of fermentation materials can be stacked in the discharging room, a heating unit is coiled inside the discharging room to promote this part of materials to be fermented beforehand and then drive the starting of the fermentation materials in the fermentation room on the upper part.

As an improvement of the above technical solution, the fermentation furnace further comprises a fire pestle composed of pestles with tips and used for making airflow through holes inside the fermentation pile, wherein, the fermentation pile automatically generates heat to drive gas in the airflow through holes to rise, and a negative pressure is generated at the lower part, so that external air enters the discharging room and then enters the airflow through holes, and thus continuously supplementing the fermentation pile.

As an improvement of the above technical, solution, the furnace hook is composed of hook fins being of a “7”-shaped structure; the furnace hook is provided with a plurality of hook fins side by side, a plurality of grid seams of the grate can be simultaneously scraped by artificial and manual operation; more further, the side-by-side hook fins of the furnace hook are fixed on a spindle, and the spindle is driven by external mechanical power to rotate and simultaneously scrape the plurality of grid seams of the grate to deal with a scene of large-scale fermentation treatment.

As an improvement of the above technical solution, the fire pestle is provided with a plurality of pestles side by side, and a row of airflow through, holes can be made by artificial and manual operation once; more further, the plurality of pestles of the fire pestle can be arrayed in a square matrix and fixed on a power arm, and the power arm is driven to up and down move by external mechanical power; lots of airflow through holes can be formed once to deal with a scene of large-scale fermentation treatment.

As an improvement of the above technical solution, the appearance of the fermentation furnace is of a cylinder shape or a square column shape; a heat preservation layer is provided outside the fermentation furnace.

Further, the top cover covers on the fermentation room to reduce the disputation of heat; an air pulling chimney is also fixed on the top cover; the air pulling chimney is equipped with an airflow regulator which regulates the volume of the airflow from the upper part.

As an improvement of the above technical solution, the width of the grid seam of the grate is based on a fact that the fermentation materials can, be supported not to drop; the cross section of the grid of the grate is of a triangular shape, so that the grid seam is of a trapezoid shape with a wide top and a narrow bottom.

The fermentation materials of the present fermentation furnace are, put into the fermentation room from the upper part to be fermented in the fermentation room, the fermented materials become loose to drop into the discharging room below and eventually are discharge to the outside, the materials in the fermentation room gradually decrease, while reduction of fermentation volume creates a new space to allow a new fermentation material to be put, thereby achieving the function of continuous fermentation and operation. This fermentation furnace sufficiently utilizes the space created by reduction of fermentation with a high space use rate, so this fermentation furnace is suitable for application scenes generating a certain quantity of rubbishes and wastes daily, such as family, residential quarter and country.

Meanwhile, this fermentation furnace also uses a natural method to achieve a balance between oxygen supply and heat preservation of the fermentation pile, and even in a cold environment, there is little external energy required to start. The whole fermentation pile is in the working state of high temperature fermentation, and each part of materials undergoes a process including preheating, high-temperature fermentation and medium-low temperature fermentation; both ventilation and heat preservation are present in the whole process; this fermentation furnace is short in fermentation period, rapid in treatment speed and also suitable for application scenes which have huge volume and need to be treated as soon as possibly, such as sludge from, a sewage treatment plant and oily sludge from an oil field.

According to further aspect of the present application, provided is a treatment system of an aerobic microorganism fermentation pile for large-scale organic solid wastes. The treatment system comprises: a large-size fermentation pile, which is formed by stacking fermentation materials and is a round-shaped pile, a trapezoid-shaped pile or a windrow-shaped pile; the width and height of the large-size fermentation pile are not limited by a fact that the fermentation pile can not be too large in order to ensure ventilating conditions;

a steel structure or brick-concrete structure, which is used for supporting the weight of the large-scale fermentation pile, the steel structure or brick-concrete structure comprising uprights, beams, purlines and, rafters, wherein, the uprights are perpendicular to the ground; the beams are horizontally and transversely erected on the top ends of two uprights: the purlines are horizontally placed on the two beams, and arrayed at an equal distance perpendicular to the beams; the rafters are horizontally placed on the two purlines and arrayed at an equal distance perpendicular to the, purlines, so as to form various small checks; the raffers are covered with straws, mats or silk screens, and the fermentation materials are filled on the straws, mats or silk screens to construct the fermentation pile; the size of the check and the thick density of the mat or silk screen or the mesh of the silk screen are based on a fact that a large amount of fermentation materials are not leaked; the height of the upright is based on a fact that a space built under the fermentation pile meets ventilation and a few amount of leaked materials are easy to take out; the suspended space supported by the steel structure or the brick-concrete structure under the fermentation materials is the ventilating passage which supplies the whole fermentation pile with oxygen; the fermentation materials at the periphery of the ventilating passage naturally drop and are sealed, and an air inlet pipe is pre-embedded or an air inlet is left to be communicated with the outside; the ventilating passage space can be above or under the ground;

a ventilating system, which comprises the ventilating passage provided at the bottom of the large-scale fermentation pile and vertical airflow through holes upwardly and downwardly penetrating through the fermentation pile, wherein, the upper parts of the vertical airflow through holes are communicated with the outside, and the lower parts thereof are communicated with the ventilating passage; the vertical airflow through holes are formed by downwardly drilling the up and down penetrated holes from the top of the, fermentation pile using a pestle or other tools, or a pipe with grids or ventilating gaps on a pipe wall is pre-embedded.

Further, the treatment system of an aerobic microorganism fermentation pile for large-scale organic solid wastes further comprises a heat preservation system which comprises a greenhouse built outside the large-size fermentation pile, or a heat preservation wall considering both heat preservation and a material fixing effect is arranged on the vertical surface of the fermentation pile.

Further, the ventilating system further comprises transverse airflow through holes, the transverse airflow through holes being formed by laying the fermenting materials and plant ventilating materials at intervals in the process of building the pile until the materials reach a preset height, wherein, a ventilating layer formed by the plant ventilating materials in the horizontal direction is the transverse airflow through hole. The transverse airflow through holes can also be achieved by drilling holes crossed and communicated with or close to the vertical airflow through holes in the horizontal direction with a tool.

Further, the greenhouse comprises a support skeleton and a hermetical shelter covering thereon; an air outlet pipe is arranged in the middle of the side surface of the greenhouse; the shelter drops on the ground and is sealed with the ground, and the air inlet pipe pre-embedded in the ventilating passage penetrates through the shelter to extend to the outside.

Further, the hermetical shelter adopts a double-layer structure whose middle is filled with air to achieve a double-layer heat preservation effect.

Further, the heat preservation system further comprises a second greenhouse to achieve the double-layer heat preservation effect.

Further, a heating device is arranged in the greenhouse is internally provided with a heating device, the heating device being a heater installed in the ventilating passage or hot vapor introduced into the ventilating passage, so that the materials at the bottom of the fermentation pile are heated and fermented at first and then the starting of the whole fermentation pile is driven.

Further, the heat preservation wall can be formed by connecting heat preservation boards, or a hollow wall is built using a brick-concrete material to surround the vertical periphery of the fermentation pile, thereby playing effects a heat preservation and material fixation at the same time.

Further, the treatment system, further comprises a percolate collecting and absorbing device, the percolate collecting and absorbing device comprising a grade and a cofferdam provided on the fermentation ground in advance; an auxiliary fermentation material for absorbing water is arranged at the percolate collecting position in the cofferdam to absorb the percolate to participate in the next-batch fermentation, thereby preventing the percolate from overflowing and smelling.

Further, the treatment system further comprises a hard block water chopping pretreatment device, the hard block water chopping pretreatment device comprising a connected container which is of a cylinder shape on the upper part and of a cone shape on the lower, part. A screen is arranged in the middle of the hard block water chopping pretreatment device. The cone-shaped container is provided with a discharging hole for cutting the materials. The vertical rotation shaft is mechanically driven by external power. Water is injected into the container, and the, hard block material is chopped in water.

Further, the treatment system further comprises an original site pre-fermentation treatment system, the original site pre-fermentation treatment system comprising holes similar to the airflow through holes or the ventilating passage structure in the pile with a tool on the original site of the waste solid pile; loose fermentation materials are put in the hole to drive the solid wastes to be fermented in advance, thereby playing roles of reduction, improvement of material properties and shortening of treatment time for formal treatment in the most easy and economic manner.

This fermentation treatment system is designed special for large-scale organic solid waste microorganism fermentation, comprising a large-size fermentation pile, a ventilating system, a heat preservation system, a percolate collecting and absorbing device and water chopping pretreatment device, wherein, the ventilating system can ensure the supply of oxygen in the fermentation pile; after the fermentation is started, a gas in, the pile is heated and rises along the vertical airflow through hole, a negative pressure is formed on the lower part, and the external air enters the ventilating passage from an, air inlet pipe and then enters the vertical airflow through holes and the transverse airflow through holes to be transported to various positions of the fermentation pile.

Different from the existing technology, the fermentation pile of this treatment system is the large-size fermentation pile, particularly meaning that the width and height, of the fermentation pile are not limited by a fact that the fermentation pile can not be too large in order to ensure traditional ventilating conditions and only depend on limitation of site and processing tools, and meanwhile its shape is, not limited and can adopt a cone shape, a trapezoid shape or a windrow shape, and is especially suitable for aerobic microorganism fermentation of large-scale organic solid wastes.

The heat, preservation system can allow heat from the fermentation pile to rest on an air layer between the surface of the fermentation pile and the greenhouse so as to reduce the loss of heat, and meanwhile capture sun energy in the daytime to heat the fermentation process to accelerate the starting of the fermentation.

The percolate collecting and absorbing device converges the percolate into one place utilizing the existing ground besides a preset manner. An auxiliary fermentation material for absorbing water can be placed at the percolate converging position to absorb the percolate to participate in the next-batch fermentation, thereby preventing overflowing and smelling.

The water chopping pretreatment device can chop large materials in water so that the materials are sufficiently mixed to improve the efficiency of fermentation. Especially for large, viscous and hard materials, they are hopped in water at first, and then immediately mixed with other fermentation materials, thereby avoiding the materials to be adhered together.

This fermentation treatment system adopts a convenient and practical structure, pre-ferments large-scale organic solid wastes and then stacks a large-size fermentation pile, a balance is formed between oxygen supply and heat preservation of the fermentation pile by virtue of a natural method, and the whole fermentation pile is in a working state of high temperature fermentation. The fermentation treatment system is short in fermentation period, quick in treatment speed, and extremely suitable for microorganism fermentation treatment of large-scale organic solid wastes.

### DESCRIPTION OF THE DRAWINGS

FIG. 1 is a flowchart of an aerobic microorganism fermentation method considering both ventilation and heat preservation according to embodiments, of the disclosure;

FIG. 2 is a sectional structure view of an aerobic microorganism fermentation device considering both ventilation and heat preservation according to one embodiment of the disclosure;

FIG. 3 is a sectional structure view of an aerobic microorganism fermentation device considering both ventilation and heat preservation according to another embodiment of the disclosure;

FIG. 4 is a sectional structure view of an aerobic microorganism fermentation device considering both ventilation and heat preservation according to another embodiment of the disclosure;

FIG. 5 is a structural diagram of a ventilating pipe of an aerobic microorganism fermentation device considering both ventilation and heat preservation according to, one embodiment of the disclosure;

FIG. 6 is a structural diagram of a ventilating pipe of an aerobic microorganism fermentation device considering both ventilation and heat preservation according to another embodiment of the disclosure;

FIG. 7 is a structural diagram of an aerobic microorganism fermentation furnace capable of continuously operating according to one embodiment of the disclosure;

FIG. 8 is a structural diagram of a furnace hook of an aerobic microorganism fermentation furnace capable of continuously operating according to one embodiment of the disclosure;

FIG. 9 is a structural diagram of a fire pestle of an aerobic microorganism fermentation furnace capable of continuously operating according to one embodiment of the disclosure;

FIG. 10 is a structural diagram of an aerobic microorganism fermentation treatment system for large-scale organic solid wastes according to another embodiment of the disclosure;

FIG. 11 is a structural diagram of a pretreatment device of an aerobic microorganism fermentation treatment system for large-scale organic solid wastes according to one embodiment of the disclosure;

FIG. 12 is a structural diagram of a connection pole of an aerobic microorganism fermentation treatment system for large-scale organic solid wastes according to one embodiment of the disclosure;

FIG. 13 is a structural diagram of a hollow joint of an aerobic microorganism fermentation treatment system for large-scale organic solid wastes according to one embodiment of the disclosure;

FIG. 14 is a structural diagram of a grounded joint, of an aerobic microorganism fermentation treatment, system for large-scale organic solid wastes according to one embodiment of the disclosure;

FIG. 15 is a structural diagram of an aerobic microorganism fermentation treatment system for large-scale organic solid wastes according to another embodiment of the disclosure.

### DETAILED DESCRIPTION OF PREFERRED EMBODIMENTS

Next, the microorganism fermentation method and device of the disclosure will be explained and described in detail in combination with accompanying drawings.

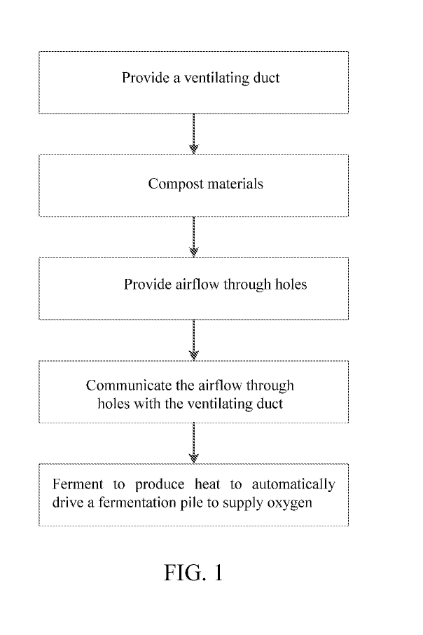

As shown, in FIG. 1, it shows a fermentation flow procedure of the microorganism fermentation method considering both ventilation and heat preservation according to one embodiment of the disclosure. The microorganism fermentation method comprises the following steps: providing a suspended space under a fermentation pile in advance so that fermentation materials are lifted above the ground, wherein, the suspended space, as a ventilating passage, is communicated with external air; stacking the fermentation materials on the ventilating passage to construct a fermentation pile; making, a plurality of up and down penetrated vertical airflow through holes on the material pile, wherein the tipper parts of the vertical airflow through holes are communicated with the outside, and the lower parts thereof arc communicated with, the ventilating passage; the vertical airflow through holes are formed by downwardly drilling up and down penetrated holes from the top of the fermentation pile by virtue of a tool, or a pipe with grids or ventilating, gaps on pipe walls, is pre-embedded, such as floral pipe for digging a well; and fermenting the materials to produce heat so as to automatically drive external air to supply the fermentation pile with oxygen, wherein, required air volume is automatically regulated without an external energy source so that the fermentation pile is stable in a high-temperature fermentation state. In the disclosure, the activity of microorganisms warms the materials, and air in the vertical holes is, heated and rises to, drive external air to be supplemented from the ventilating passage at the bottom of the fermentation pile to enter the vertical holes, so that oxygen is continuously and evenly supplied to various parts of the fermentation pile. A relationship between the air supplementation speed and the fermentation temperature is that if the fermentation temperature is high, the rapid the air supplementation is, which is enough to meet a need of oxygen required by fermenting a large amount of microorganisms at high temperature. If the fermentation temperature is low, air supplementation is slow, facilitating that the fermentation pile preserves heat. A solid ventilating pipe can be inserted into the airflow through hole, and the ventilating pipe is used to prevent the materials from collapsing in the process of fermentation to block the airflow through holes. Foreign objects can not he added in the airflow through holes, instead, airflow through holes communicated with the ventilating holes are drilled in the material pile. The disclosure uses a completely natural method to solve a conflict between oxygen supply and heat preservation of the fermentation pile so that the fermentation pile reaches an optimal system balance. The fermentation pile can rapidly achieve high-temperature fermentation, thereby accelerating the treatment speed and reducing the treatment time.

In other embodiments of the disclosure, the aerobic microorganism fermentation method considering both ventilation and heat preservation further comprises: achieving control on the whole fermentation progress through the drilling sequence of the vertical airflow ventilating holes, including timing, a pore diameter, quantity and location. In another embodiments, the aerobic microorganism fermentation method considering both ventilation and heat preservation further comprises: providing a heat preservation layer outside the fermentation pile, so as to facilitate preservation of heat to achieve high temperature treatment without affecting supply of oxygen.

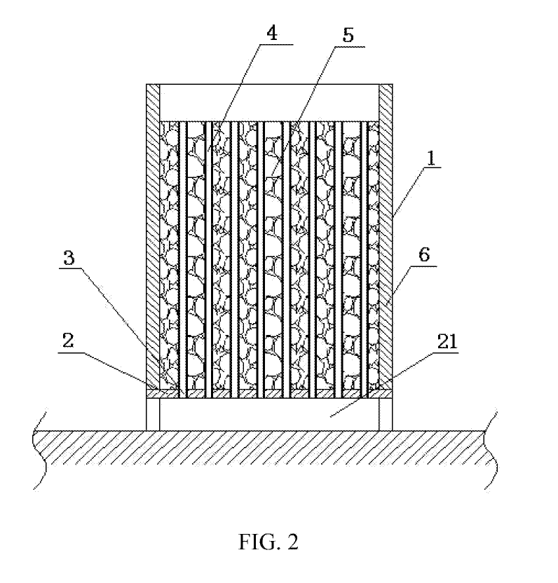

In combination with FIG. 2, it shows a structural diagram of an microorganism fermentation device according to one embodiment of the disclosure. Disclosed is microorganism fermentation device considering both ventilation and heat preservation, comprising a fermentation chamber 1, wherein, the bottom of the fermentation chamber is provided with a bottom board 2, a ventilating passage 21 communicated with the outside is arranged under the bottom board, a plurality of ventilating holes 3 are formed in the bottom board, the fermentation chamber is internally provided with airflow through holes for ventilating the material pile, and the airflow through holes pass through the ventilating holes on the bottom board to be communicated with the ventilating passage.

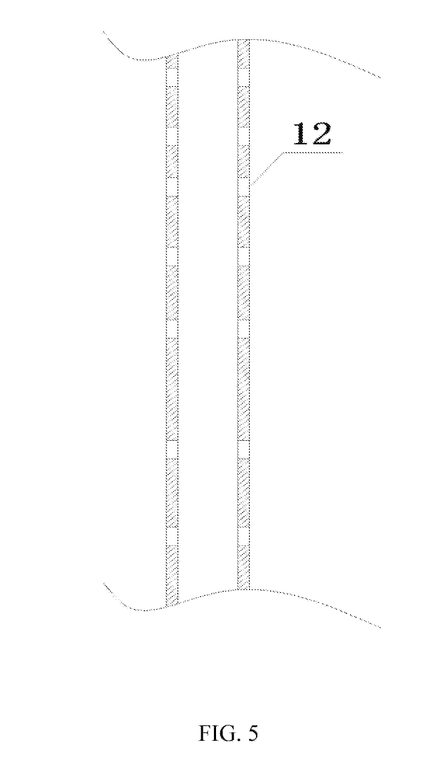

In combination with FIG. 5, the airflow through hole comprises a ventilating pipe 4 the side wall of which is provided with a plurality of through holes 12.

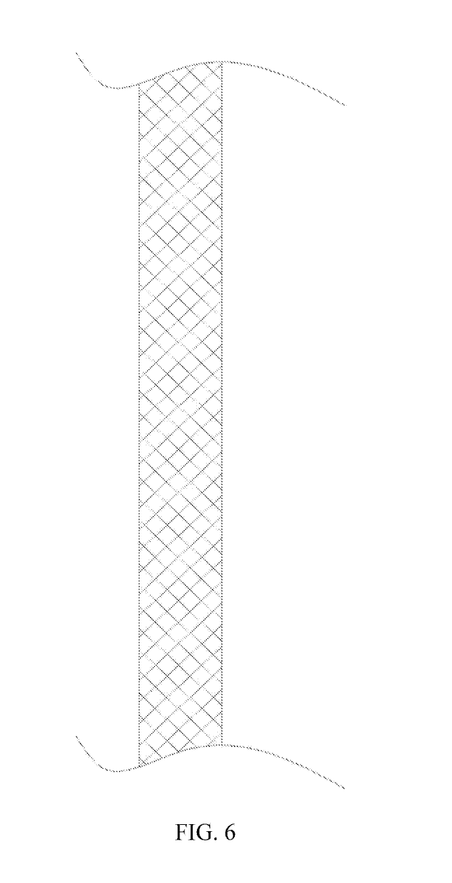

As shown in FIG. 6 the airflow through hole comprises a ventilating:pipe 4 the side wall of which is of a grid structure.

The solid pipe is taken out from the airflow through hole after the discharging hole is processed, and the up and down penetrated air hole is only remained.

In the disclosure, the air through hole adopts the ventilating pipe to, prevent the materials from being collapsed to block the airflow through hole. The ventilating pipe can adopt a steel pipe provided with a plurality of ventilating holes on a side wall or other pipes, and can also adopt a pipe with grids on a side wall. If the pipe with the ventilating holes is adopted, the support effect is good, and if the pipe with grids is adopted, the ventilating effect is good.

The airflow through holes penetrate through the material pile. In this embodiment, the airflow through holes penetrate through the material pile, airflow rapidly flows and evenly exchanges oxygen with the material at the circumference of the airflow through holes, and fermentation progress of the material is even.

The outer side of the fermentation chamber is provided with a heat preservation layer 6. Due to adoption of the heat preservation layer, heat dissipation is reduced, the fermentation temperature in the fermentation chamber is improved, the fermentation speed is accelerated, and the fermentation time is reduced. The heat preservation layer does not affect the oxygen supply of the fermentation pile.

The bottom board is of a grill or bar structure. In this technical solution, the support effect of the grill or bar is good in support effect, large in ventilating hole area and large in air intake.

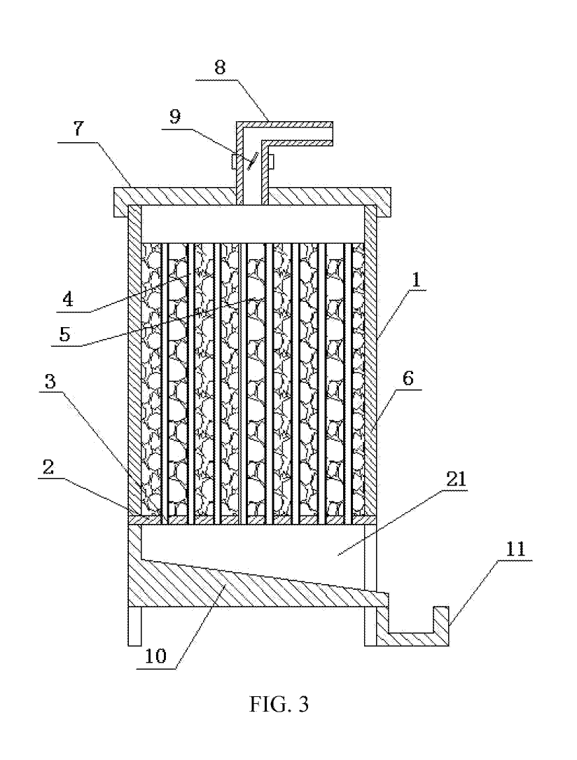

In combination with FIG. 3, the fermentation chamber is provided with a top cover 7. The top cover is communicated with an air pulling chimney 8, and the air pulling chimney is equipped with an airflow regulator 9. In this embodiment, adoption of the top cover is beneficial to maintaining the temperature in the heat preservation chamber and ensuring the need of the fermentation temperature; the top cover is provided with the air pulling chimney, which can accelerate the gas in the fermentation chamber to be discharged outwardly. The passing through rate of airflow is controlled by adopting the airflow regulator.

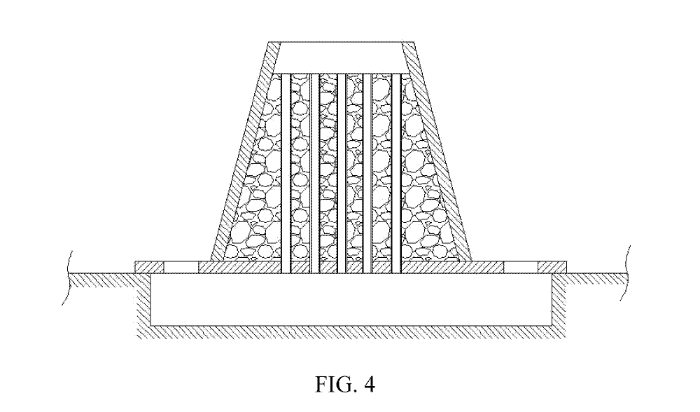

In combination with FIG. 4, a part of the fermentation chamber can be made into a trapezoid-shaped strip windrow on the ground. In FIG. 4, the ventilating passage is arranged either on the ground or under the ground as long as it is communicated with the outside. Airflow can sufficiently supply the material pile with oxygen.

In combination with FIG. 3 and FIG. 4, in the disclosure, the excess water of, the raw materials in the fermentation chamber or the excess water generated in the process of fermentation passes through the vent holes on the bottom board and then drips into the ventilating passage to flow into the liquid collecting groove 11 installed under the fermentation chamber side in FIG. 3 or a pit in FIG. 4. In FIG. 3, the plane of the liquid collecting groove is lower than the bottom of the fermentation chamber, and meanwhile the fermentation chamber is connected with the liquid collecting groove by an inclined plane, so as to facilitate flow of the liquid into the liquid collecting groove. In order to facilitate installation of the liquid collecting groove, a base 10 with the inclined plane is installed at the bottom of the fermentation chamber, the liquid collecting groove is installed at one side, close to the lower part, of the inclined plane of the base. The liquid collecting groove can also be directly installed at the bottom of the fermentation chamber, the bottom of the fermentation chamber is equipped with the base, and the base is provided with the inclined plane extending to the liquid collecting groove, so as to reduce an occupied area of the liquid collecting groove being installed on the side lower part.

In combination with FIG. 2 and FIG. 3, in the disclosure, the base board is moveably connected to the bottom of the fermentation chamber. The bottom board 2 is designed as being detachable, thereby achieving a continuous operation function of feeding on the upper part and discharging on the lower part.

The bottom of the fermentation pile of the disclosure is lifted higher than a peripheral plane, and air can be supplemented from the bottom; a large amount of vertical holes similar to honeycomb briquettes are downwardly drilled from the upper part of the fermentation material; a heat preservation measure is added at the periphery of the vertical surfaces of the fermentation pile. In the fermentation process of the disclosure, after the temperature of the fermentation pile is raised, air in the airflow through holes is heated and then moves upwardly, a negative pressure is formed at the bottom, and surrounding air enters the ventilating passage and then flows into the airflow through holes, so that new oxygen supplementation is brought, microorganisms obtain more oxygen, movement is accelerated, more heat is generated, and more air supplementation is brought, thereby forming continuous air flow.

In the disclosure, airflow is driven to automatically flow completely depending on the self heat of the fermentation pile, so that oxygen is continuously and evenly supplied to various parts of the fermentation pile. The volume of airflow is automatically regulated according to the heat of the fermentation pile, so as to achieve optimal system balance. The heat preservation measure of the outer layer is beneficial to formation of a higher temperature of the fermentation pile without affecting oxygen supply. (A relationship between the air supplementation speed and the fermentation temperature is that the fermentation temperature is high, and air supplementation is quick, which is enough to supplement oxygen required for high temperature fermentation activity of a large amount of microorganisms; the fermentation temperature is low, and air supplementation is slow, facilitating preservation of heat of the fermentation pile. In the disclosure, a conflict between the oxygen supply and the heat preservation of the fermentation pile is solved by using a completely natural method so that the fermentation pile achieves an optimal system balance; the fermentation pile can rapidly achieve high-temperature fermentation, thereby accelerating the treatment speed and reducing the treatment time.)

In the fermentation process of the disclosure, as shown in FIGS. 3 and 4, excess water of the material is downwardly permeated out of the material at the early stage of fermentation and stored in the liquid collecting groove, and the fermentation pile reaches a temperature suitable for microorganism reproduction. In the process of fermentation, water stored in the liquid collecting groove can be appropriately supplemented to the fermentation pile again. For the extremely humid and permeability-poor material, the disclosure can also consider both two aspects, namely, ventilation and heat preservation.

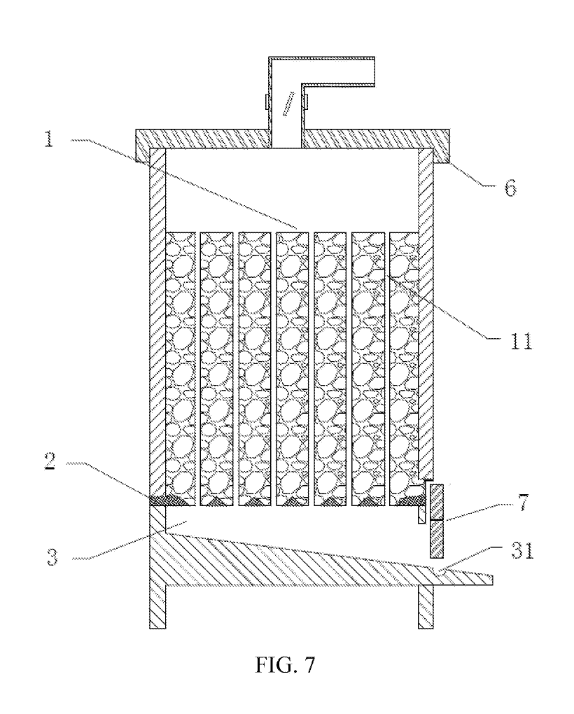

FIG. 7 shows an aerobic microorganism fermentation furnace capable of continuously operating according to one embodiment of the disclosure. Referring to FIG. 7-FIG. 9, the aerobic microorganism fermentation furnace mainly comprises a top cover 76, a fermentation room 71, a grate 72 and a discharging room 73, wherein, the appearance of the fermentation furnace is of either a cylinder shape or a square column shape, and a heat preservation layer is provided outside the aerobic microorganism fermentation furnace.

Particularly, the vertical surfaces of the fermentation room 71 are sealed, the upper part of the fermentation room is opened, the fermentation material enters from the upper part to be stacked in the fermentation room 71 to form the fermentation pile.

One side of the discharging room 73 is provided with the discharging hole, the discharging hole upwardly extends from the bottom of the discharging room 73 over the grate 72 to a space above the bottom of the fermentation room 71; the discharging hole is equipped with a flapper 77; after the flapper is opened, a fermentation situation at the bottom of the fermentation room 71 can be checked above the grate 72, and a material which is large in caking ability and difficult to drop can be subjected to assistant treatment; discharging is performed under the grate 72, the flapper 77 is covered and properly sealed after discharging, thereby achieving a function of regulating the input of air from the lower part.

For convenience, the flapper 77 can be divided into two parts, a part above the grate 72 is not often opened and sealed using adhesive tapes; a part under the grate 72 is frequently opened, and properly sealed by filling the gaps of the part under the grate 72 with the fermentation material. The bottom surface of the discharging room 73 is inclined at a certain angle from the inner to the outer, the outer side of the discharging room is provided with a water collecting groove 731 for collecting water permeated out of the fermentation pile, and meanwhile the inclined structure allows discharging to save more labor. The width and height of the discharging hole are provided based on a fact that they are suitable for discharging.

In the cold environment, the discharging room 73 is internally provided with a temporary heater for heating air entering the fermentation pile to an indoor, temperature in winter to promote the rapid starting and rapid fermentation of the fermentation pile. The temporary heater can adopt water heating, electric heating and the like and can be placed on a slide trolley so as to facilitate movement and disassembly. A shelter is placed on the temporary heater to avoid the fermentation material to drop into the heater; or a part of fermentation material can be stacked in the discharging room, a heating unit is coiled inside the discharging room 3 to promote this part of material to be fermented at first and then to drive the starting of the fermentation material in the fermentation room 71 above.

The grate 72 is arranged between the fermentation room 1 and the discharging room 73, is of a bar structure and is used for blocking the material in the fermentation room 71 above not to drop.

The width of the grid seam of the grate 72 is based on a fact that the fermentation material is supported not to drop, the material which is put in the fermentation room 71 at the early stage of operation can partially drop into the discharging room 73, and after the feeding is completed, the material can stay in the fermentation room 71 above as long as the material is slightly squeezed and, cannot drop again without an external force, and the material dropping into the discharging room 73 is gently drawn out to be put into the fermentation room 71. There is no special limitation on the grid of the grate 72, and the cross section of the grid is optimally of a triangular shape so that the grid seam is of a trapezoid shape with a wide upper part and a narrow lower part.

In this disclosure, this fermentation furnace allows a balance between the oxygen supply and the heat preservation of the fermentation pile, and even in the cold environment, there is a little opportunity for starting required external energy. On this basis, the fermentation material is put into the fermentation room 71 from the upper part to be fermented in the fermentation room 71, and then drops into the discharging room 73 below after being fermented and finally discharged to the outside. The material in the fermentation room 71 above is gradually declined, while reduction of fermentation creates a new space to allow a new fermentation material to be put, and hereby the aerobic microorganism fermentation furnace, with feeding on the upper part and discharging on the lower part, capable of continuously operating. That is to say, the fermentation furnace of, the disclosure further has a continuous operation capability on the basis of considering both ventilation and heat preservation.

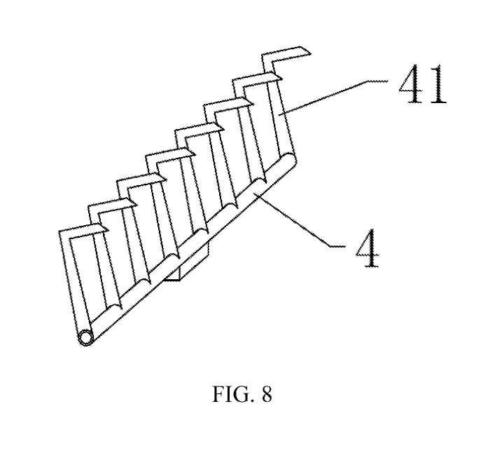

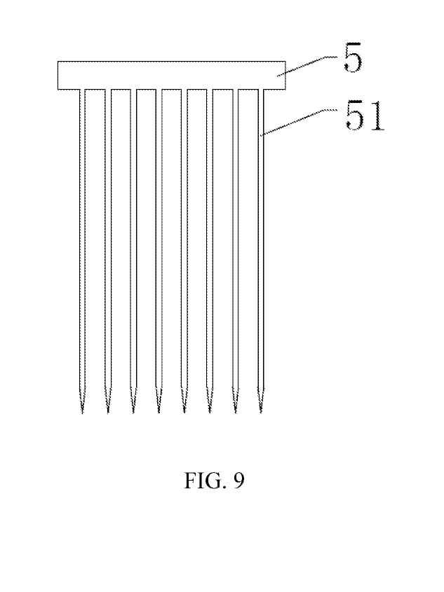

Referring to FIG. 8 and FIG. 9, this fermentation furnace further comprises a furnace hook 74 for discharging and a fire pestle 75 making airflow through holes 711 inside the fermentation pile, wherein, the furnace hook 74 is composed of hook fins 741 being of a “7”-shaped structure. When it is needed to perform discharging, the loose fermented material is scraped with the furnace hook 74 along the grid seams of the grate 72 so that the material on the bottom layer drops into the discharging room 73.

The fire pestle 75 is composed of pestles 751 with tips and used for making a plurality of airflow through holes 711 communicated with the discharging room 73 inside the fermentation pile, and then the fermentation pile automatically generates heat to drive gas in the airflow through holes to rise and generate a negative pressure on the lower part, so that external air enters the discharging room 73 and then enters the airflow through holes 711 so as to continuously supply the fermentation pile.

In partial embodiments of this fermentation furnace, in, order to improve the working efficiency, a plurality of hook fins 741, whose structure is similar to that of a rake, are arranged on the furnace hook 74 side by side, and a plurality of grid seams of the grate 72 can be scraped through artificially and manually operation once; more further, the, side-by-side hook fins 741 can be fixed on a spindle, and the spindle can be driven by external mechanical power to rotate and simultaneously scrape the materials on the plurality of grid seams. Similarly, the fire pestle 75 is side by side provided with a plurality of pestles 751 whose structure is similar to that of a big comb, and a row of airflow through holes 711 can be made by artificial and manual operation once; more further, the plurality of pestles 751 can be arrayed in a square matrix and fixed on a power arm; the power arm is driven to up and down move by external mechanical power, and lots of airflow through holes 711 can be made once.

The top cover 76 covers the top of the fermentation room 71 to reduce the disputation of heat; an air pulling chimney is also fixed on the top cover; the air pulling chimney is equipped with an airflow regulator to regulate the volume of the airflow from the upper part.

The fermentation material of the present fermentation furnace is put into the fermentation room 71 from the upper part and fermented in the fermentation room 71, the fermented material becomes loose to drop into the discharging room 73 below and eventually discharge to the outside; the material in the fermentation room 71 above gradually decreases, while reduction of fermentation creates a new space to allow a new fermentation material to be put, thereby achieving the aerobic microorganism furnace, with feeding on the upper part and discharging on the lower part, capable of, continuously operating. This fermentation furnace sufficiently utilizes the space created by reduction of fermentation with a high space use rate, so this fermentation furnace is very suitable for application scenes generating a certain quantity of rubbishes and wastes daily, such as families, residential quarters and countries.

Meanwhile, this fermentation furnace allows a balance between oxygen supply and heat preservation of the fermentation pile, and even in the cold environment, there is little external energy source required to start. The whole fermentation furnace is in the working state of high temperature fermentation; each part of material undergoes a process including preheating, high-temperature fermentation and medium-low temperature fermentation; both ventilation and heat preservation are present in the whole process; the fermentation furnace is short in fermentation period, rapid in treatment speed and also suitable for application scenes which have huge mass and need to be treated as soon as possibly, such as sludge from a sewage treatment plant and oily sludge from an oil field.

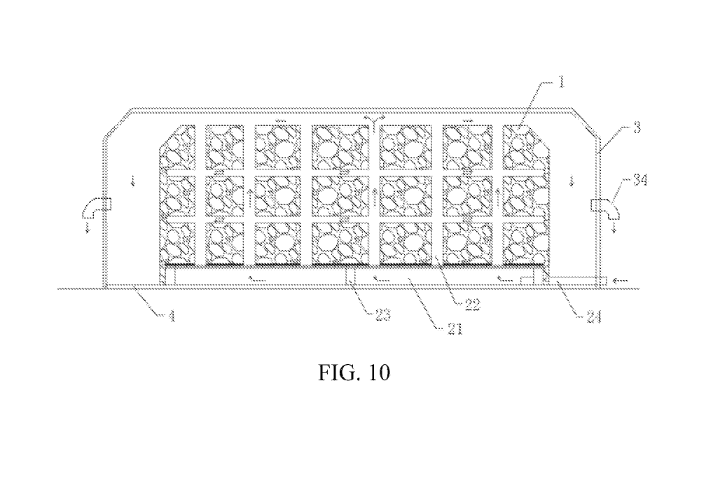

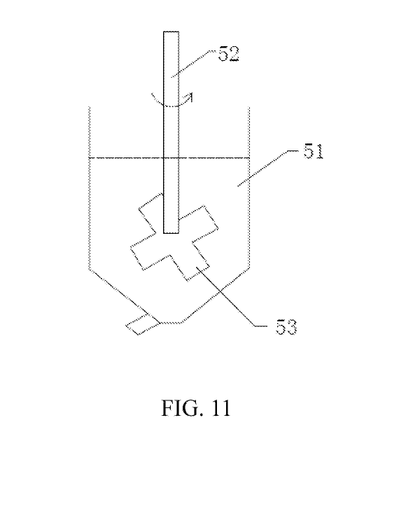

As shown in FIG. 10, it shows an aerobic microorganism fermentation treatment system for large-scale organic solid wastes according to one embodiment of the disclosure. Referring to FIG. 10 and FIG. 11, the aerobic microorganism fermentation treatment system for large-scale organic solid wastes primarily comprises a large-size fermentation pile 81, which is formed by stacking pretreated fermentation materials, here, the large-size fermentation pile 81 means that the width and height of the fermentation pile are not limited by a fact that the fermentation pile cannot, be too large in order to ensure ventilating conditions and only depend on limitation of sites and processing tools, and the shape is not limited, and thus the large-size fermentation pile can be a cone-shaped pile, a trapezoid-shaped pile or a windrow-shaped pile;

a ventilating system, which comprises the ventilating passage 821 provided at the bottom of the large-size fermentation pile 81 and airflow through holes 822 penetrating through the interior of the large-size fermentation pile 81. The ventilating system can ensure the supply of oxygen in the fermentation pile. After fermentation is started, the gas in the pile is heated to rise along the vertical airflow through holes, and a negative pressure is formed on the lower part, and the external air enters the ventilating passage 821 from an air inlet pipe 824 and then enters the vertical airflow through holes and the transverse airflow through holes to be transported to various parts of the fermentation pile.

Where, the ventilating system also comprises a steel structure or brick-concrete structure 823 for supporting the weight of the large-size fermentation pile 81, the steel structure or brick-concrete structure 823 comprising uprights, beams, purlines and rafters, wherein, the uprights are vertical to the ground, the beams are horizontally and transversely erected on the top ends of two uprights, the purlines are horizontally placed on the two beams and arrayed at an equal distance perpendicular to the beams, and the rafters are horizontally placed on the two purlines and arrayed at an equal distance perpendicular to the purlines so as to form small checks; the raffers are covered with straws, mats or silk screens, and then the fermentation material is filled on the straws, mats or silk screens to build the fermentation pile. The size of the check and the thick density of the mat or silk screen or the mesh of the silk screen is based on a fact that a large amount of fermentation materials are not leaked, the height of the upright is based on a fact that the space built under the fermentation pile meets ventilation and a few amount of leaked materials are easy to take. The ventilating passage 821 is the suspended space supported by the steel structure or the brick-concrete structure 823 to supply the whole fermentation pile with oxygen, the four sides of the ventilating passage 821 are sealed by naturally dropped fermentation material, and an air inlet pipe 824 is pre-embedded or an air inlet is left to be communicated with the outside. The ventilating passage space can be above or under the ground.

In the microorganism fermentation treatment system of the disclosure, a convenient and practical structure is adopted, the large-scale organic solid wastes are pretreated at first so that a balance is formed between oxygen supply and heat preservation of the fermentation pile. The microorganism fermentation treatment system is designed special for large-scale organic solid waste microorganism fermentation. A large-size fermentation pile 81 is stacked on the basis of pretreatment of large-scale organic solid wastes, and the whole fermentation pile is in a working state of high-temperature fermentation. Both ventilation and heat preservation are present in the whole process. The microorganism fermentation treatment system is short in fermentation period and rapid in treatment speed, and is extremely suitable for microorganism fermentation of large-scale organic solid wastes.

It can be seen from FIG. 10 that the airflow through hole 822 comprises a transverse airflow through hole and a vertical airflow through hole. A fermentation material and a plan ventilating material are laid in the fermentation pile at intervals until the fermentation pile achieves a predetermined height, wherein, the plant ventilating material forms the transverse airflow through hole, the vertical airflow through hole is formed downwardly via penetration by virtue of the pestle or other tools from the top of the fermentation pile, for example, when the height of the fermentation pile is suitable for artificial operation, the pestles are arrayed in a matrix and driven by an mechanical arm to be downwardly press, lots of vertical airflow through holes can be formed once, while for a higher fermentation pile, a flexible drill can be used to replace the pestle. The lower ends of the vertical airflow through holes are communicated with the ventilating passage 821.

The greenhouse 83 of the heat preservation system comprises a support skeleton and a hermetical shelter covering thereon, wherein, an air outlet pipe 834 is arranged in the middle of the side surface of the greenhouse 83, the shelter drops on the ground and is sealed with the ground, and the air inlet pipe 824 penetrates through the shelter to extend to the outside.

The heat preservation system can allow heat from the fermentation pile to rest on an air layer between the, surface of the fermentation pile and the greenhouse 83 so as to reduce loss of heat, and meanwhile can capture sun energy in the daytime to heat the fermentation process to accelerate the starting of fermentation.

The air layer of the above greenhouse 83 is not communicated with the ventilating passage 821 and the air inlet pipe 824. Airflow in the air layer flows in a sequence of the air inlet pipe 824, the ventilating passage 821, the airflow through hole 822, the air layer and the air outlet pipe 834. In order to facilitate artificial regulation, airflow regulation valves can be arranged both in the air inlet pipe 824 and the air outlet pipe 834.

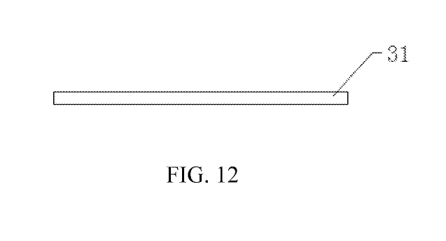

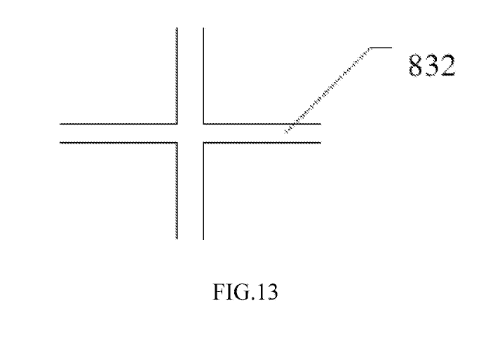

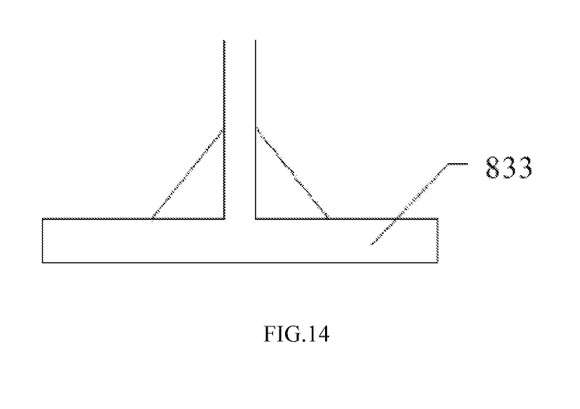

Referring to FIGS. 12-14, the above greenhouse 83 is distanced from the surface of the fermentation pile by about 50 cm and arranged outside the fermentation pile. An air heat preservation layer is formed between the greenhouse and the fermentation pile. The support skeleton is composed of connection poles 831 and joints, the joint including a hollow joint 832 and a grounded joint 833, wherein, the hollow joint 832 connects connection poles 831 in four directions together, the grounded joints 833 allow the vertical connection poles to be fixed on the ground and not to shake left and right, the lower ends of the ground connection joints 833 can be fixed by virtue of heavy materials on the ground so that the connection poles 831 above do not shake left and right, and the connection poles 831 and the joints can be fixed through bolts or chucks.

This embodiment further comprises a percolate collecting and absorbing device 84 which is arranged under the large-size fermentation pile 81. Particularly, the percolate collecting and absorbing device 84 can be a slope and a cofferdam that are predesigned at the periphery of the fermentation ground, or the percolate collecting and absorbing device 84 allows the percolate to be converged into a place by utilizing the known ground and terrain. An auxiliary fermentation material for absorbing water is placed at the percolate converge position to absorb the percolate to participate in the next-batch fermentation, thereby preventing the percolate from overflowing and smelling.

Referring to FIG. 11, a waster chopping pretreatment device comprises a water container 851, a vertical spindle 852 arranged above the water container and a horizontal spiral cutting knife 853 installed at the lower end of the vertical spindle 852. The waster chopping pretreatment device is, used for cutting a large material in water before the fermentation pile is stacked, so that the fermentation materials are sufficiently mixed. Particularly, the water container 851 is a connection container with a cylinder shape on the upper part and a cone shape on the lower part, a screen is arranged in the middle of the water container, the cone-shaped container is provided with a discharging hole for chopping the material, and the vertical spindle 852 is driven by an external power machine such as a driver.

When in operation, water is injected in the water container 851 to reach the middle lower apart of the cylinder-shaped container, and the large viscous and hard material drops into water so that it slowly drops and chopped by the horizontal spiral cutting knife 853; the small material drops into the cone-shaped container below, and the large material up and down floats to be further cut under the drive of spiral waterpower. An economic use method is as follows: the original material is directly fermented at first, the fermented material is screened after first fermentation, the large viscous and hard material is put again to be chopped, in such a way, quantity is reduced to a certain extent.

The water chopping pretreatment device can chop the large materials in water so that the materials are sufficiently mixed to improve the efficiency of fermentation. Especially for the large viscous and hard material, it is chopped in water at first and then mixed with other fermentation materials, thereby avoiding the materials to be adhered together.

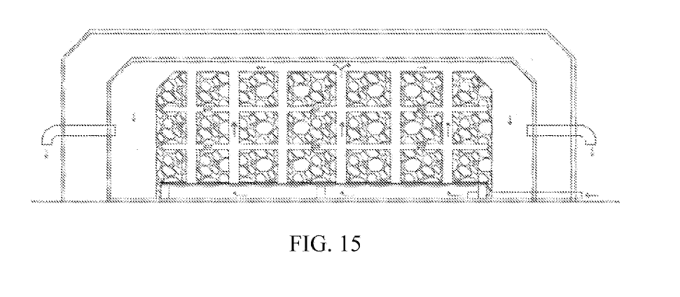

Referring to FIG. 15, when in an extremely cold region, a double-layer sealing material containing an air layer is used for heat preservation, or a second layer of greenhouse is arranged outside the greenhouse to achieve a double-layer heat preservation effect similar to double-layer glass. For a material which is extremely diluted and difficult to shape, it can be fixed adopting a heat preservation wall. The heat preservation wall comprises a wall body formed by connecting heat preservation boards, or a hollow wall body built by using a brick-concrete material, to surround the, periphery of the vertical surface of the fermentation pile thereby simultaneously playing a role of heat preservation and material fixation.

In order to improve the efficiency of fermentation treatment, a heating device is arranged in the greenhouse. The heating device is a heater installed in the ventilating passage as the existing device, or hot vapor introduced into the ventilating passage, so as to warm and ferment the material at the bottom of the fermentation pile and then further drive the starting of the whole fermentation pile.

The fermentation treatment system also comprises a pretreatment fermentation system, the pretreatment fermentation system comprising an extremely large-scale solid waste pile as well as airflow-like through holes and a ventilating passage-like structure formed therein. The pretreatment fermentation system ferments extremely large-scale solid waste pile in advance in the most convenient and economic manner to play roles of reduction, improvement of material property and shortening of treatment time for formal treatment.

For example, for the extremely large-scale existing solid waste pile (such as rubbish hill), it can be directly fermented according to the device principle of the, present utility. A particular operation is as follows: before formal fermentation is performed, positions are measured on the solid waste pile in row and line to make a vertical hole array, auxiliary fermentation materials and strains are put in each vertical hole, and then a plurality of horizontal passages are drilled on the lower part of the solid waste pile by aligning at lines or rows of the holes to penetrate through various vertical holes, so as to uniformly supply the whole solid waste pile with oxygen, upholders are appropriately added into the vertical holes and horizontal passages, and thus the vertical airflow-like through holes and the ventilating passage-like structure are built on the solid waste pile. The solid waste pile is, fermented in advance in the most convenient and economic manner, thereby playing roles of reduction, improvement of material property and shortening of treatment time.

The fermentation treatment device is designed special for large-scale organic solid waste microorganism fermentation. It adopts a convenient and practical structure, pretreats large-scale organic solid wastes and then composts a large-size fermentation pile. By using the fermentation treatment device, a balance is formed between oxygen supply and heat preservation of the fermentation pile by virtue of a natural method, and the whole fermentation pile is in a working state of high-temperature fermentation. Both ventilation and heat preservation are present in the whole process. The fermentation treatment device is short in fermentation period and rapid in treatment speed, and is extremely suitable for microorganism fermentation of large-scale organic solid wastes

The above contents are only for describing embodiments in detail but cannot be regarded as limiting the implementation scope of the present disclosure. Modifications and improvements made hereby are all within the scope of the disclosure.

We claim:

1. An aerobic microorganism fermentation method considering both ventilation and heat preservation, comprising the following steps:
providing a suspended space under a fermentation pile in advance so that fermentation materials are lifted above the ground, wherein, the suspended space, as a ventilating passage, is communicated with external air;
stacking the fermentation materials on the ventilating passage to build the fermentation pile;
making a plurality of up and down penetrated vertical airflow through holes on the fermentation material pile, wherein, the upper parts of the vertical airflow through holes are communicated with the outside, and the lower parts thereof are communicated with the ventilating passage; the vertical airflow through holes are formed by downwardly drilling up and down penetrated holes from the top of the fermentation pile by virtue of a tool, or a pipe with grids or ventilating gaps on pipe walls is pre-embedded; and
fermenting the materials to produce heat so as to automatically drive external air to supply the fermentation pile with oxygen, wherein, the required air volume is automatically regulated without an external energy source so that the fermentation pile is stable in a high-temperature fermentation state.

2. The method according to claim 1, further comprising: providing a heat preservation layer outside the fermentation pile, so as to facilitate preservation of heat to achieve high temperature treatment without affecting supply of oxygen.

3. The method according to claim 1, wherein, the fermentation pile is internally provided with transverse airflow through holes which perpendicularly penetrate through the vertical airflow through holes, so as to enhance the diffusion effect of the entered air; the transverse airflow through holes are achieved by horizontally laying plant ventilating materials at different layer heights of the fermentation pile, or by drilling holes crossed and communicated with or close to the vertical airflow through holes with a tool in a horizontal direction.

4. A treatment system of an aerobic microorganism fermentation pile for large-scale organic solid wastes, comprising:
a large-size fermentation pile, which is formed by stacking fermentation materials and is a round-shaped pile, a trapezoid-shaped pile or a windrow-shaped pile, wherein, the width and height of the large-size fermentation pile are not limited by a fact that the fermentation pile can not be too large in order to ensure ventilating conditions;
a steel structure or brick-concrete structure, which is used for supporting the weight of the large-size fermentation pile, the steel structure or brick-concrete structure comprising uprights, beams, purlines and rafters, wherein, the uprights are perpendicular to the ground; the beams are horizontally and transversely erected on the top ends of two uprights; the purlines are horizontally placed on the two beams, and, arrayed at an equal distance perpendicular to the beams; the rafters are horizontally placed on the two purlines and arrayed at an equal distance perpendicular to the purlines, so as to form various small checks; the raffers are covered with straws, mats or silk screens, and the fermentation materials are filled on the straws, mats or silk screens to construct the fermentation pile; the size of the check and the thick, density of the mat or silk, screen or the mesh, of the silk screen are based on a fact that a large amount of fermentation materials are not leaked; the height of the upright is based on a fact that a space built under the fermentation pile meets ventilation and a few amount of leaked materials are easy to take out; the suspended space supported by the steel structure or the brick-concrete structure under the fermentation materials is the ventilating passage which supplies the whole fermentation pile with oxygen; the fermentation materials at the periphery of the ventilating passage naturally drop and are sealed, and an air inlet pipe is pre-embedded or an air inlet is left to be communicated with the outside; the ventilating passage space can be above or under the ground;
a ventilating system, which comprises the ventilating passage provided at the bottom of the large-size fermentation pile and vertical airflow through holes upwardly and downwardly penetrating through the fermentation pile, wherein, the upper parts of the vertical airflow through holes are communicated with the outside, and the lower parts thereof are communicated with the ventilating passage; the vertical airflow through holes are formed by downwardly drilling the up and down penetrated holes from the top of the fermentation pile with a pestle or other tools, or a pipe with grids or ventilating gaps on pipe walls is pre-embedded.

5. The treatment system of an aerobic microorganism fermentation pile for large-scale organic solid wastes according to claim 4, wherein, the treatment system further comprises a heat preservation system, the heat preservation system comprising a greenhouse built outside the large-size fermentation pile; the greenhouse comprises a support skeleton and a hermetical shelter covering thereon; an air outlet pipe is arranged in the middle of the side surface of the greenhouse; the shelter drops on the ground and is sealed with the ground; the air inlet pipe pre-embedded in the ventilating passage passes through the shelter to extend to the outside.

6. The treatment system of an aerobic microorganism fermentation pile for large-scale organic solid wastes according to claim 5, wherein, the hermetical shelter adopts a double-layer structure whose middle is filled with air, so as to achieve a double-layer heat preservation effect.

7. The treatment system of an aerobic microorganism fermentation pile for large-scale organic solid wastes according to claim 4, wherein, the heat preservation system further comprises a second greenhouse, so as to achieve the double-layer heat preservation effect.

8. The treatment system of an aerobic microorganism fermentation pile for large-scale organic solid wastes according to claim 4, wherein, the greenhouse is internally provided with a heating device, the heating device being a heater installed in the ventilating passage or hot vapor introduced into the ventilating passage, so as to heat and ferment the materials at the bottom of the fermentation pile at first and then drive the starting of the entire fermentation pile.

9. The treatment system of an aerobic microorganism fermentation pile for large-scale organic solid wastes according to claim 4, wherein, the treatment system further comprises a percolate collecting and absorbing device, the percolate collecting and absorbing device comprising a slope and a cofferdam provided on the fermentation ground in advance; an auxiliary fermentation material for absorbing water is put at the percolate converging position in the cofferdam to absorb the percolate to participate in the next-batch fermentation, thereby preventing the percolate from overflowing and smelling.

10. The treatment system of an aerobic microorganism fermentation pile for large-scale organic solid wastes according to claim 4, wherein, the treatment system further comprises an original site pre-fermentation treatment system, the original site pre-fermentation treatment system comprising holes similar to the airflow through holes or the ventilating passage structure in the pile with a tool on the original site of the waste solid pile; a loose fermentation material is put in the holes to drive the solid wastes to be fermented in advance, thereby playing roles of reduction, improvement of material properties and shortening of treatment time for formal treatment in the most easiest and economic manner.

11. An aerobic microorganism fermentation furnace capable of continuously operating, comprising a top cover, a fermentation room, a grate and a discharging room, wherein, the vertical surfaces of the fermentation room are sealed, the upper part of the fermentation room is opened, and the fermentation materials are put from the upper part and forms the fermentation pile therein; the fermentation pile is internally provided with several airflow through holes communicated with the discharging room below, and the grate is arranged between the fermentation room and the discharging room, is of a grid structure and is used for blocking the materials in the fermentation room above to drop; this fermentation furnace also comprises a furnace hook for discharging; the loose fermented materials are scraped with the furnace hook along the grid seams of the grate when it is needed for discharging so that the materials on the bottom layer drop into the discharging room, hereby, the aerobic microorganism fermentation furnace capable of continuously operating, in which materials are fed from the upper part and discharged from, is formed.

12. The aerobic microorganism fermentation furnace capable of continuously operating according to claim 11, wherein, one side of the discharging room is provided with a discharging hole; the discharging hole upwardly extends from the bottom of the discharging room over the grate to a space above the bottom of the fermentation room; the discharging hole is equipped with a flapper; after the flapper is opened, a fermentation situation at the bottom of the fermentation room is checked above the grate and a material which is large in caking ability and difficult to drop is subjected to assistant treatment; discharging is performed under the grate, after discharging, the flapper is properly sealed, so as to achieve a function of regulating the input of air from the lower part; the bottom surface of the discharging room is inclined at a certain angle from the inner to the outer, and a water collecting tank is arranged outside the discharging room and used for collecting water bleeding from the fermentation pile; the discharging room is also internally provided with a temporary heater for heating air entering the fermentation pile in cold winter until the indoor temperature is reached, and the rapid starting and rapid fermentation of the fermentation pile and rapid fermentation are promoted.

13. The aerobic microorganism fermentation furnace capable of continuously operating according to claim 11, further comprising a fire pestle composed of pestles with tips and used for making airflow through holes inside the fermentation pile, wherein, the fermentation pile automatically generates heat to drive gas in the airflow through holes to rise, and a negative pressure is generated at the lower part, so that external air enters the discharging room and then enters the airflow through holes, and thus continuously supplementing the fermentation pile.

14. The aerobic microorganism fermentation furnace capable of continuously operating according to claim 11, wherein, the furnace hook is composed of hook fins being of a “7”-shaped structure; the furnace hook is provided with a plurality of hook fins side by side so that a plurality of grid seams of the grate are simultaneously scraped to improve working efficiency.

15. The aerobic microorganism fermentation furnace capable of continuously operating according to claim 11, wherein, the side-by-side hook fins of the furnace hook are fixed on a spindle, the spindle is driven by external mechanical power to rotate and simultaneously scrape the plurality of grid seams of the grate to deal with a scene of large-scale fermentation treatment.

16. The aerobic microorganism fermentation furnace capable of continuously operating according to claim 13, wherein, the fire pestle is provided with a plurality of, pestles side by side, and a row of airflow through holes can be made once to improve a working efficiency.

17. The aerobic microorganism fermentation furnace capable of continuously operating according to claim 14, wherein, the plurality of pestles of the fire pestle are arrayed in a square matrix and fixed on a power arm; the power arm is driven to up and down move by external mechanical power; lots of airflow through holes can be made once to deal, with a scene of large-scale fermentation treatment.

18. The aerobic microorganism fermentation furnace capable of continuously operating according to claim 11, wherein, the appearance of the fermentation furnace is of a cylinder shape or a square column shape, and a heat preservation layer is provided outside the fermentation furnace.

19. The aerobic microorganism fermentation furnace capable of continuously operating according to claim 18, wherein, the top cover covers on the fermentation room to reduce the disputation of heat; an air pulling chimney is also fixed on the top cover; the air pulling chimney is equipped with an airflow regulator which regulates the volume of the airflow from the top.

20. The aerobic microorganism fermentation furnace capable of continuously operating according to claim 11, wherein, the width of the grid seam of the grate is based on a fact that the fermentation materials can be supported not to drop; the cross section of the grid of the grate is of a triangular shape, so that the grid seam is of a trapezoid shape with a wide top and a narrow bottom.
